(19) United States
(12) Patent Application Publication
(10) Pub. No.: US 2025/0069761 A1
(43) Pub. Date: Feb. 27, 2025

(54) INFORMATION PROCESSING DEVICE

(71) Applicant: ONLINEDOCTOR.COM CO., LTD., Tokyo (JP)

(72) Inventor: Mikihiro SUZUKI, Tokyo (JP)

(21) Appl. No.: 18/719,917

(22) PCT Filed: Oct. 21, 2022
(86) PCT No.: PCT/JP2022/039270

§ 371 Date: Jul. 17, 2024

(30) Foreign Application Priority Data
Dec. 16, 2021 (JP) 2021-204132

(51) Int. Cl. G16H 80/00; G16H 10/60; G16H 40/20

(52) U.S. Cl. CPC G16H 80/00; G16H 10/60; G16H 40/20

(57) ABSTRACT

This information processing device comprises: an acquisition means that acquires second user information from a second user terminal operated by a second user wishing for matching with a first user having a predetermined attribute; an extraction means that extracts the first user who can be matched, on the basis of the second user information; and an output means that outputs the second user information and a list of the second users who have been matched, to a terminal of the first user.

### CROSS REFERENCE TO RELATED APPLICATIONS

This is the U.S. national stage of application No. PCT/JP2022/039270, filed on Oct. 21, 2022. Priority under 35 U.S.C. § 119 (a) and 35 U.S.C. § 365 (b) is claimed from Japanese Application No. 2021-204132, filed Dec. 16, 2021, the disclosure of which is also incorporated herein by reference.

### TECHNICAL FIELD

The present invention relates to an information processing device.

### BACKGROUND ART

Technologies related to matching a patient with a medical facility have been proposed. For example, Patent Document 1 discloses a technology of sending a medical questionnaire to the patient, analyzing the completed medical questionnaire, and presenting a medical facility suitable for the patient's symptom to the patient.

### CITATION LIST

### Patent Document

Patent Document 1: Japanese Unexamined Patent Application, Publication No. 2003-030325

### DISCLOSURE OF THE INVENTION

### Problems to be Solved by the Invention

In recent years, there has been an increasing need for online consultations due to the desire to avoid medical facility-acquired infections and to find the most suitable medical facility irrespective of its location. Online consultations do not require the patient to directly visit the medical facility, and the patient can select the medical facility not only in their neighborhood but over a broader area.

However, it is not easy for the patient seeking an online consultation to quickly find a medical facility that offers online consultations among a number of medical facilities. Under these circumstances, an object of the present invention is to provide a technology that can match a person with a person having a predetermined attribute in real time.

### Means for Solving the Problems

To achieve the object described above, an aspect of the present invention is directed to an information processing device including:
an acquisition unit that acquires second user information on at least one second user who wishes to match with at least one first user having a predetermined attribute from a second user terminal operated by the second user;
an extraction unit that extracts at least one first user who is matchable based on the second user information; and
an output unit that outputs a list of the second users matched and the second user information to a terminal of the first user.

### Effects of the Invention

The present invention allows a person to match with a person having a predetermined attribute in real time.

### BRIEF DESCRIPTION OF THE DRAWINGS

FIG. 1A and FIG. 1B are views illustrating an outline of an online consultation matching system;

FIG. 2 is a view illustrating a configuration of the online consultation matching system;

FIG. 3 is a block diagram illustrating a hardware configuration of a server;

FIG. 4 is a functional block diagram illustrating an example of a functional configuration of the server;

FIG. 5 is a flowchart of an online consultation matching process;

FIG. 6 is a flowchart of an example of a notification process;

FIG. 7 is a flowchart of an example of a process of presenting a medical record;

FIG. 8A and FIG. 8B are views illustrating an example of a screen for application of the online consultation;

FIG. 9 is a view illustrating an example of a screen of a waiting room for the online consultation (for a patient);

FIG. 10A to FIG. 10C are views illustrating an example of a screen of a waiting room for the online consultation (for a medical facility); and

FIG. 11 is a view illustrating an example of a start screen of the online consultation.

### PREFERRED MODE FOR CARRYING OUT THE INVENTION

### Embodiments

### <Outline>

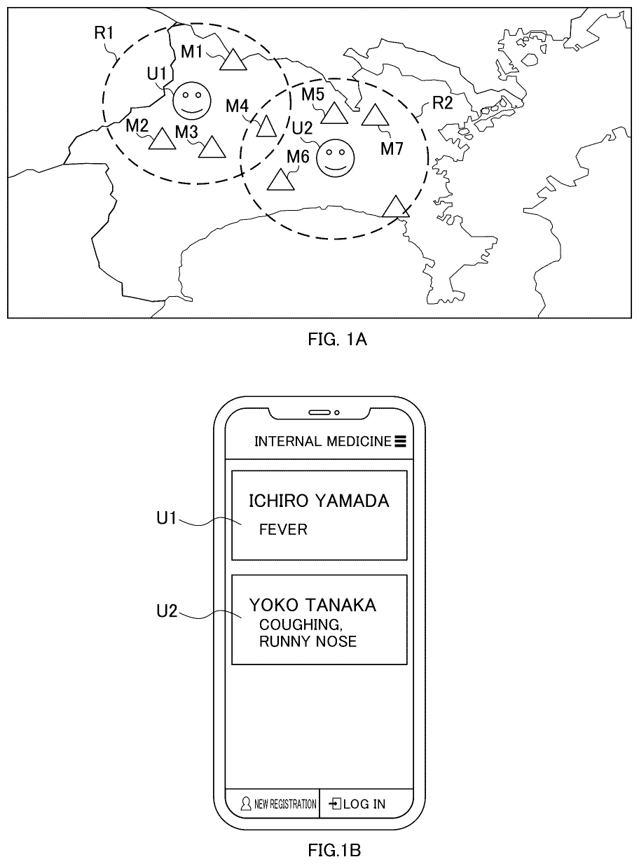

Embodiments will be described below with reference to the drawings. FIG. 1A and FIG. 1B are views illustrating an outline of an online consultation matching system of the present embodiment. In the example of FIG. 1A, the locations of users U1 and U2 are shown. The locations of medical facilities M1 to M7 are also shown. Regions R1 and R2 are areas where an online consultation is available for the users U1 and U2 (second users). In the present embodiment, the address of the user (hereinafter referred to as a user U unless otherwise specified) will be described as an example of the location of the user. However, the location is not limited to this example, and may be, for example, the user's current location, the location of the user's workplace, or any location specified by the user U. In the present embodiment, the address of the medical facility will be described as an example of the location of the medical facility. However, the location is not limited to this example, and may be, for example, the current location of a doctor (a first user having a predetermined attribute). In the present embodiment, the area where the online consultation is available is an area within a predetermined distance (e.g., 30 km) from the user's location (a location indicated by location information on the user). In the example of FIG. 1A, the medical facilities M1 to M4 can offer the user U1 the online consultation. The medical facilities M4 to M7 can offer the user U2 the online consultation.

When the user wishes to have face-to-face consultation in the medical facility, the user generally visits the facility directly or makes a reservation by phone or through a website before the visit. The user who directly visits the medical facility needs to wait in a waiting room in the medical facility until other patients waiting to be seen have finished their consultations. That is, waiting time arises depending on the other patients, and the user needs to wait in the medical facility until the waiting time passes. The user who has made a reservation by phone visits the medical facility in consideration of time the user waits for the consultation (e.g., time left until the reservation time informed by the medical facility). The user who made a reservation through the website is informed of the number of waiting patients in the reserved medical facility and visits the medical facility when the user's turn is coming soon.

The number of medical facilities that offer online consultations has been increasing in recent years. Thus, the user does not need to directly visit the medical facility and can receive the consultation not only at nearby medical facilities (e.g., within walking distance) but also at medical facilities located over a wider area. However, it often takes a long time for the user to make a reservation in consideration of how crowded the medical facilities offering the online consultation are. The patients are supposed to have the online consultation with a so-called “primary care doctor”, and there are various conditions for first-time patients to meet. This is a constraint on those who seek the online consultation.

In view of these circumstances, the online consultation matching system of the present embodiment allows the user to inform multiple medical facilities in an area where the online consultation is available for the user that the user is waiting for the consultation and places the user in a waiting state. For example, the system informs the medical facility M4 in FIG. 1A that the users U1 and U2 are waiting for the consultation as shown in FIG. 1B. Thus, the user who wishes to have the online consultation can be easily matched with the available medical facility in real time. That is, the user can have the desired online consultation more quickly. The display shown in FIG. 1B representing the patients waiting for the online consultation can be recognized as a virtual waiting room. In the following description, the function related to the waiting room for the online consultation of the present embodiment will be referred to as a “waiting room function”.

The online consultation matching system of the present embodiment can store a medical record of the user in a predetermined storage in advance and can present the record to the doctor if necessary (e.g., at the doctor's request). This allows a first-time patient to have the online consultation.

Further, the online consultation matching system of the present embodiment can offer online counseling (e.g., services not covered by public medical insurance) with healthcare professionals such as doctors and nurses in all parts of the country irrespective of the area where the online consultation is available. If necessary, the online counseling in progress can be switched to the online consultation for further examination and prescription when the counseling is conducted at the medical facility in the area where the online consultation is available. For the online counseling, not only the doctors and the patients, but companies can register and manage the patients (users). For example, a company can offer the online counseling as one of employee benefits and register the employees. Alternatively, an insurance provider such as an insurance company can offer the online counseling as one of services using the system of the present embodiment. As an example of a partner company, a welfare facility such as a nursing home can register residents requiring nursing care for the consultation using a system of the online consultation or counseling in case of emergency. The online consultation matching system of the present embodiment will be described in detail below.

### <System Configuration>

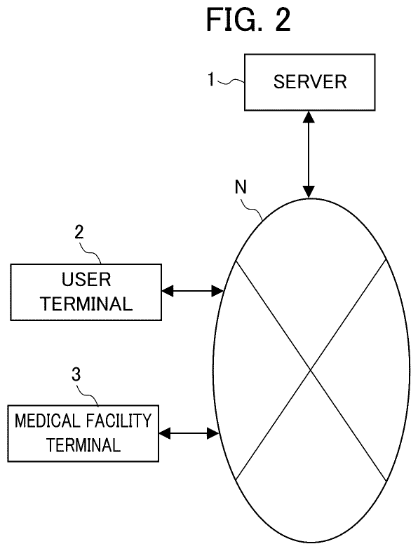

FIG. 2 is a view schematically illustrating a system configuration of the online consultation matching system of the present embodiment. The online consultation matching system of the present embodiment includes a server 1 (an information processing device) that performs processing, a user terminal 2 (a second user terminal) operated by a user (e.g., a patient), and a medical facility terminal 3 (a first user terminal) operated by a person in charge in a medical facility (e.g., a doctor). The server 1, the user terminal 2, and the medical facility terminal 3 are mutually connected via a predetermined network N such as the Internet. The user terminal 2 does not have to be a terminal owned by the patient and may be a terminal of a person on behalf of an elderly patient, for example.

### <Hardware Configuration>

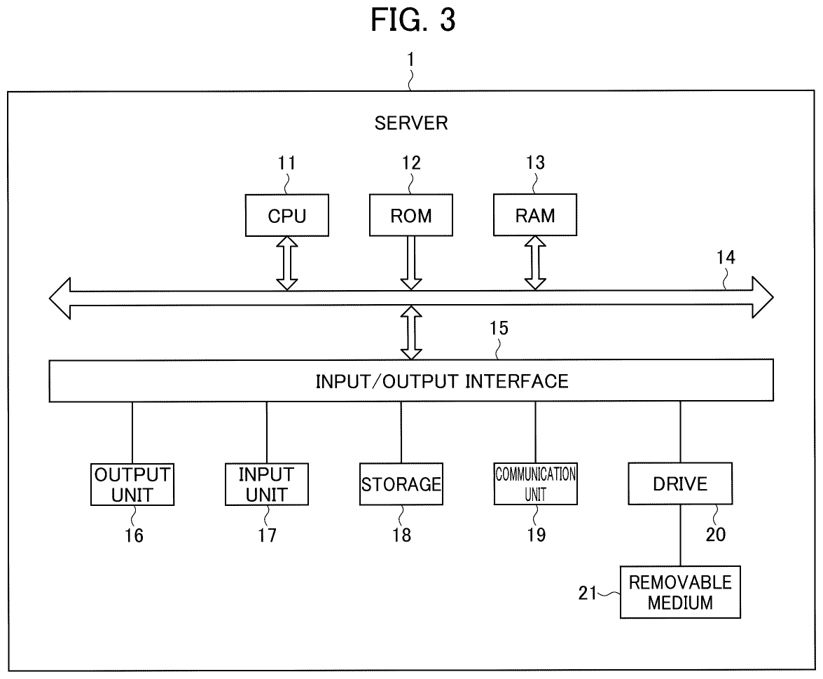

FIG. 3 is a block diagram illustrating a hardware configuration of the server 1 of the present embodiment. The server 1 includes a central processing unit (CPU) 11, read only memory (ROM) 12, random access memory (RAM) 13, a bus 14, an input/output interface 15, an output unit 16, an input unit 17, a storage 18, a communication unit 19, and a drive 20.

The CPU 11 performs various processes in accordance with a program stored in the ROM 12 or a program loaded from the storage 18 into the RAM 13. The RAM 13 appropriately stores data that the CPU 11 requires to perform the processes. The CPU 11, the ROM 12, and the RAM 13 are mutually connected via the bus 14. The input/output interface 15 is also connected to the bus 14.

The output unit 16, the input unit 17, the storage 18, the communication unit 19, and the drive 20 are connected to the input/output interface 15. The output unit 16 is constituted of a display, a speaker, or any other suitable device and outputs various pieces of information as an image or audio. The input unit 17 is constituted of a keyboard, a mouse, or any other suitable device and receives various types of information. The storage 18 is constituted of a component such as a hard disk or dynamic random access memory (DRAM) and stores various types of data. The communication unit 19 communicates with other devices via the network N including the Internet.

A removable medium 21, such as a magnetic disk, an optical disk, a magneto-optical disk, or semiconductor memory, is suitably attached to the drive 20. A program read from the removable medium 21 by the drive 20 is installed in the storage 18 as needed. The removable medium 21 can also store various data stored in the storage 18, just like the storage 18.

Although not shown, the user terminal 2 and the medical facility terminal 3 each have the hardware configuration shown in FIG. 3.

### <Functional Configuration>

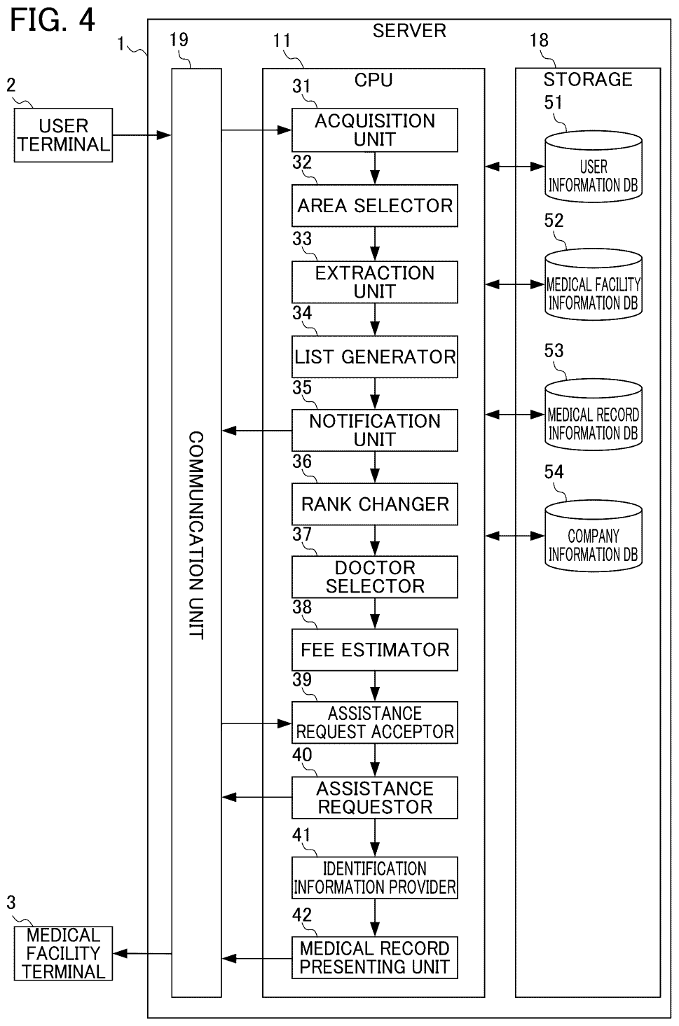

FIG. 4 is a functional block diagram illustrating an example of the functional configuration of the server 1 of the present embodiment.

When the CPU 11 of the server 1 runs, an acquisition unit 31, an area selector 32, an extraction unit 33, a list generator 34, a notification unit 35, a rank changer 36, a doctor selector 37, a fee estimator 38, an assistance request acceptor 39, an assistance requestor 40, an identification information provider 41, and a medical record presenting unit 42 function.

The acquisition unit 31 acquires user information from the user terminal 2. The acquired user information is stored in a user information DB 51. The user information includes, for example, location information on the user, information about departments, medical form information, medical record information, health insurance card information, and medical facility information. The location information on the user indicates the current location of the user in the present embodiment. The department is a clinical department that the user wishes to visit for the online consultation. The medical form information is information previously inputted in a medical questionnaire by the user and includes, for example, patient's information (such as name, gender, date of birth, phone number, and e-mail address), body temperature, symptoms, time of onset of the symptoms, and any special needs (e.g., designation of the medical facility and the doctor). The medical form information may be prepared by using a predetermined system (e.g., AI (artificial intelligence) medical form system) run by a company operating the matching system or collaboration with other company. In the present embodiment, the medical record information (a patient chart) is medical information about patient's medical progress and includes medical details, the results of past tests (such as the results of a blood test and a checkup), a patient referral document (a so-called referral letter), patient chart information, a record of hospital admission, a record of hospital discharge, patient chart information at the hospital admission, patient chart information on hospital visits, and information on prescribed drugs (a prescription). The acquired medical record information is stored in a medical record information DB 53 described later. The health insurance card information is, for example, an image of a health insurance card taken by the user. The acquisition unit 31 may check the expiry date of the health insurance card based on the acquired health insurance card information and may inform the user or the medical facility if the card is expired. The health insurance card may have no expiry date depending on the institution that issued the card. In this case, the expiry date is not checked. The medical facility information is information about the medical facility registered by an administrator of the matching system or a person in charge in the medical facility. Examples of the medical facility information includes information about the name, address, and phone number of the medical facility, and information about the “waiting room function” of the system such as availability of the waiting room, available departments, working hours (days of the week and periods of time), an e-mail address for notification, the names of available doctors, an e-mail template for registration confirmation, and payment information (e.g., account information on the payee). The acquisition unit 31 stores the acquired medical facility information in a medical facility information DB 52. In the present embodiment, the acquisition unit 31 can accept a priority request for a priority consultation and designation of the medical facility and the doctor for the online consultation from the user terminal 2. The doctor is obliged to give the patient a hardcopy of the patient chart information in response to the patient's request. In the current regional medical cooperation system, the doctor is supposed to input (e.g., upload) the patient chart information in the system. However, the patient chart information can be acquired when the patient requests the doctor to provide the patient chart information and registers the information to the system in person. Examples of the user information may include information on the results of various tests including image diagnosis, information on the checkup results, information on drug prescription, information on a nursing summary at the hospital admission, information on a nursing summary at the hospital discharge, information on a nursing summary during hospitalization, a patient referral document, information on various administrative notifications prepared for the patient by the doctor, allergy information, drug contraindication information, various opinions written by an attending doctor, genetic information, information on clinical trial participation, an Individual Number Card, a driver's license, medical coupons issued by an administrative body, information on a nursing care-related certification issued by the administrative body, information on tests related to physical development and psychological state conducted by the administrative body, various orders by the doctor, certificates by the doctor, meal information including images, a passport, a record of physical activities, vital information from a wearable terminal, the results of self-measurement of blood pressure, the results of self-measurement of blood sugar, information on self-measurable vitals, anamnesis, and a family history.

The area selector 32 determines the consultation available area where the online consultation is available for the user based on the user information. For example, an area within a predetermined distance (e.g., 30 km) or a predetermined travel time from the user's current location (the location indicated by the user's location information) is determined as the consultation available area. The area selector 32 does not determine the consultation available area by the distance on the map alone and can take the travel by transportation such as a train and an express way into account for the determination of the consultation available area. The location as a reference for the predetermined distance or travel time is not limited to the current location of the user and may be the location of a home or office of the user, a home of a person requiring nursing care, a nursing home, or a nursery school.

The extraction unit 33 extracts the medical facilities that can offer the online consultation in real time in the consultation available area based on the medical facility information stored in the medical facility information DB. For example, the extraction unit 33 extracts the medical facilities that can offer the online consultation in real time and are located in the consultation available area based on the consultation available area and the addresses of the medical facilities. When no medical facility that can offer the online consultation in real time is found in the consultation available area, the extraction unit 33 may give notification to the medical facilities in the consultation available area (e.g., after a predetermined time). If any of the medical facilities that can offer the online consultation can respond to the notification, the extraction unit 33 notifies the patient (the user terminal 2) that the patient can have the consultation and ask the patient if the patient waits for the consultation. If the patient waits, the medical facility (the medical facility terminal 3) is notified of the presence of the waiting patient. When no medical facility is available, the request for the online consultation may be sent as a request for the online counseling to the medical facilities out of the consultation available area. The online counseling may be done by other healthcare workers than the doctors, such as nurses. The extraction unit 33 may extract the medical facilities that can offer the online counseling in real time based on the medical facility information. The extraction unit 33 may extract the medical facilities that can solve the problem of the user (the patient) based on the user information (e.g., the medical form information). The extracted medical facilities are presented (proposed) to the user by, for example, the notification unit 35 described later. The function of extracting the medical facilities that can solve the problem of the user (the patient) may be implemented on, for example, various types of online counseling services, online consultation search services, and face-to-face consultation search services. Thus, the medical facility suitable for the symptom of the user can be presented (proposed) to the user.

The list generator 34 generates a list of users waiting for the online consultation in the consultation available area where the extracted medical facilities are located. The list generated by the list generator 34 is, for example, a list given to the medical facility terminal 3. In the example of FIG. 1(A), the list generator 34 generates, for example, a list of the “user U1” alone as the list of the users waiting for the online consultation to be given to the medical facilities M1 to M3. The list generator 34 also generates, for example, a list of the “user U1” and the “user U2” as the list of the users waiting for the online consultation to be given to the medical facility M4. In this case, the list also includes information on the waiting ranks of the “user U1” and the “user U2”. For example, the list generator 34 generates a list of the “user U2” alone as the list of the users waiting for the online consultation to be given to the medical facilities M5 to M7. The list generator 34 may generate a list of users waiting for the online counseling when a request for the online counseling by the user is accepted.

The notification unit 35 notifies each of the medical facility terminals 3 used by one or more medical facilities extracted of the list and the user information (e.g., the department and the name and symptom of the user). The notification unit 35 also notifies the user terminal 2 of the department, a registration number, the number of waiting patients (the waiting rank of the user in the list of the users waiting for the online consultation in the medical facility in the consultation available area). The notification unit 35 may notify the user that the consultation for the user will occur soon when the number of waiting patients is reduced to a predetermined number (e.g., five) or less, i.e., when the user's waiting rank reaches a predetermined rank. For example, the notification unit 35 may notify a message such as “your turn is coming soon” when the number of waiting patients is reduced to the predetermined number or less. The notification unit 35 may notify that the user is in a virtual waiting room (a second waiting room) different from the virtual waiting room initially displayed (the first waiting room) when the number of waiting patients is reduced to the predetermined number or less. The notification unit 35 may notify the medical facility terminal 3 of the estimated medical fee for each of the users. The estimation of the medical fee will be described later together with the description of the fee estimator 38. The notification unit 35 may notify the user terminal 2 of the number of doctors currently available. When there are no doctors available or when the doctors are no longer available, the notification unit 35 may show a message such as “yy doctors will be available in xx hours”. If there are no available doctors, the notification unit 35 may show a message, for example, “Would you like to call for available doctors?” and make a call if the patient wishes. The waiting time for the consultation (xx hours described above) may be estimated by using an identifier generated by a machine learning algorithm. More precise information may be displayed on the user terminal 2 by learning information on how many patients are waiting and how many doctors are available. The notification unit 35 may display on the user terminal 2 that the number of available doctors varies depending on the time of day. Thus, it is assumed that the patient changes time to have the consultation depending on the situation. The notification unit 35 (an output unit) may display on the medical facility terminal 3 information on busy time of day when many patients are waiting. Thus, it is assumed that market forces allow the doctor to adjust time to have the consultation according to the busy time. When there are no clinics open, the notification unit 35 may display on the user terminal 2 the text “There are no clinics open and you have left the waiting room. The reception of your area/department will open at hh:mm on MM, DD, YYYY”. When the patient leaves the waiting room, the storage 18 stores data of the medical questionnaire and the history of the medical form. When the request for the online counseling by the user is accepted, the notification unit 35 may notify the medical facility terminals 3 used by the medical facilities available for the online counseling extracted by the extraction unit 33 of the list of the users waiting for the online counseling and the user information.

In response to the priority request, the rank changer 36 changes the waiting rank of the user who made the priority request higher than the waiting ranks of the other users who made no priority request, if above priority request is made. For example, suppose that users A, B, and C are the first, second, and third users in the waiting rank of the same area (medical facility). In this case, the user A is usually the first person to have the online consultation. However, if the user C makes the priority request, for example, the rank changer 36 changes the waiting rank of the user C higher than the waiting ranks of the users A and B. The method of above priority request is not particularly limited. The priority request can be made by any method, for example, by pressing a “priority request” button (premium button) displayed on the user terminal 2. In this case, a predetermined system fee may be charged to the user who made the priority request. When the doctor regards that the user should be given priority based on the medical form information (e.g., the symptom), the user (patient) may have the priority consultation, irrespective of the user's priority request.

The doctor selector 37 determines the doctor in charge of the online consultation in response to the user's designation of the doctor. For example, when the user wishes to have the online consultation with a predetermined doctor D, the user can designate the doctor of any of the medical facilities extracted by the extraction unit 33. In this case, a predetermined system fee may be charged to the user who designated the doctor. When the doctor designated by the user is available for the online consultation in real time, the doctor selector 37 determines the doctor as the doctor in charge of the online consultation. For example, the notification unit 35 may also notify that the doctor is designated when showing the list to the medical facility terminal 3 of the corresponding medical facility. Likewise, the doctor selector 37 may determine the medical facility in charge of the online consultation in response to the user's designation of the medical facility. Specifically, the list is generated and notified to one or more facilities designated by the user among the medical facilities extracted by the extraction unit 33.

The fee estimator 38 estimates the medical fee for the consultation of the user based on the user information. For example, if the storage 18 stores a medical fee table of the departments and symptoms of the users and corresponding average medical fees, the fee estimator 38 can calculate the medical fee based on the medical fee table. When the list is transmitted to the medical facility terminal 3, the medical facility that meets a predetermined condition (e.g., paid the predetermined system fee) can select the patient to see online from the notified list irrespective of the user's waiting rank.

The assistance request acceptor 39 accepts a request to assist the user in preparing the medical form information on the user's symptom. The medical form information is important for the consultation of the user. Thus, the medical form information is assumed to be more accurate (e.g., suitably includes information necessary for the consultation) when a healthcare worker (e.g., a nurse, a pharmacist, or a person who experienced medical office work) helps the patient to input the information than when the patient inputs the information alone. It is often unclear for the user (e.g., the patient) what to write as the medical form information. Thus, the system can accept the user's request for the assistance in preparing the medical form information via the assistance request acceptor 39. The assistance request acceptor 39 may inform the medical facility terminal 3 of information indicating that which licensed worker among the healthcare workers gave the assistance.

When the user's assistance request is accepted as described above, the assistance requestor 40 requests the healthcare worker (e.g., via a healthcare worker terminal operated by the healthcare worker) to assist the user in preparing the medical form information. The healthcare worker is not limited to the person belonging to the medical facility that offers the online consultation. For example, the assistance may be requested to the healthcare workers in all parts of the country who can give the assistance in real time irrespective of the consultation available area. When the available healthcare worker responds to the request, the healthcare worker can assist the user in preparing the medical form information via chat or a web conference system.

The identification information provider 41 gives the identification information to the medical record acquired from the user. For example, the identification information provider 41 issues a two-dimensional code corresponding to the medical record acquired from the user and transmits the code to the user terminal 2. This allows the user terminal 2 to display the two-dimensional code on the user terminal 2 when desired by using an application corresponding to the system.

The medical record presenting unit 42 (presenting unit) refers to the medical record information DB 53 to present the medical record corresponding to the identification information based on the two-dimensional code (identification information) during the online consultation. For example, when the user shows the two-dimensional code to the doctor during the online consultation via the web conference system, the doctor can read the two-dimensional code using the application corresponding to the system on the medical facility terminal 3. The medical record presenting unit 42 shows (presents) the medical record corresponding to the two-dimensional code to the doctor (on the medical facility terminal 3). This allows the doctor to see the medical record of the user for the online consultation. The medical record presenting unit 42 may present the medical record using the mail function (at the patient's request). A person in charge of the medical facility (doctor) can access the system of the present embodiment to read the medical record information on the patient. The medical record presenting unit 42 may limit the function related to the presentation of the medical record. For example, the medical record may be presented on condition that a predetermined fee (a system fee) is paid by the doctor (the medical facility) when it is a predetermined number of times or more the doctor (the medical facility terminal 3) has requested the presentation of the medical record. Whether the predetermined fee is paid may be stored in, for example, the medical facility information DB 52. The function is also limited when a new patient has requested the presentation a predetermined number of times or more. The restriction is lifted when the predetermined fee is paid.

The storage 18 of the server 1 shown in FIG. 4 includes the user information DB 51, the medical facility information DB 52, the medical record information DB 53, and a company information DB 54.

The user information DB 51 stores, for example, the location information, departments, medical form information, medical record information, health insurance card information, and medical facility information on the user. As described above, the user information DB 51 stores, information on the results of various tests including image diagnosis, information on the checkup results, information on drug prescription, information on a nursing summary at the hospital admission, information on a nursing summary at the hospital discharge, information on a nursing summary during hospitalization, a patient referral document, information on various administrative notifications prepared for the patient by the doctor, allergy information, drug contraindication information, various opinions written by an attending doctor, genetic information, information on clinical trial participation, an Individual Number Card, a driver's license, medical coupons issued by an administrative body, information on a nursing care-related certification issued by the administrative body, information on tests related to physical development and psychological state conducted by the administrative body, various orders by the doctor, certificates by the doctor, meal information including images, a passport, a record of physical activities, vital information from a wearable terminal, the results of self-measurement of blood pressure, the results of self-measurement of blood sugar, information on self-measurable vitals, anamnesis, and a family history.

The medical facility information DB 52 stores information about the name, address, and phone number of the medical facility, and information about the “waiting room function” of the system such as availability of the waiting room, available departments, working hours (days of the week and periods of time), an e-mail address for notification, the names of available doctors, an e-mail template for registration confirmation, and payment information (e.g., information on payment of a predetermined fee).

The medical record information DB 53 stores medical details, the results of past tests (such as the results of a blood test and a checkup), a patient referral document (a so-called referral letter), patient chart information, a record of hospital admission, a record of hospital discharge, patient chart information at the hospital admission, patient chart information on hospital visits, and information on prescribed drugs (prescriptions).

The company information DB 54 stores information on the patients (users) registered by a company for the online counseling. Examples of the company include general companies that offer the online counseling as one of employee benefits, insurance companies, and nursing homes.

### <Processes>

### «Online Consultation Matching Process»

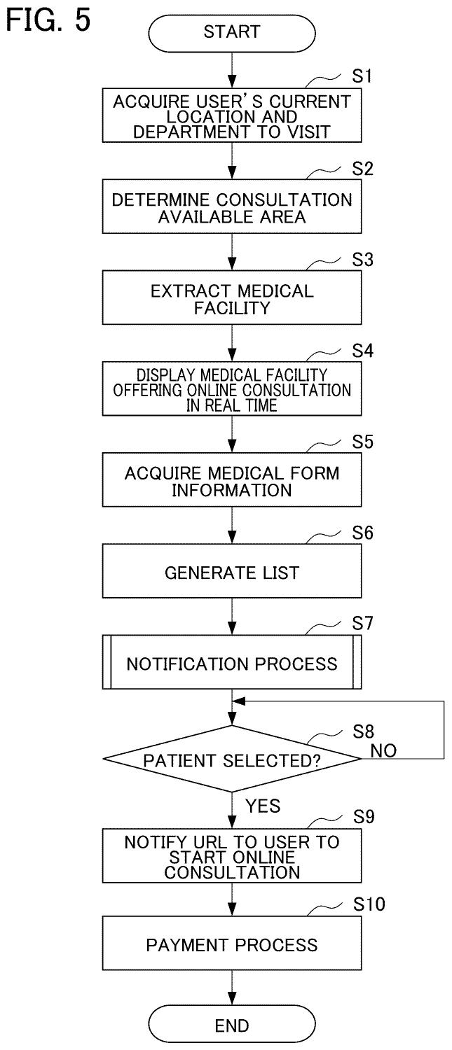

FIG. 5 is a view illustrating an example of an online consultation matching process of the present embodiment. Specifically, it is an example of a series of processes performed when the user has the online consultation.

In Step S1, the acquisition unit 31 acquires, for example, the current location of the user and the department that the user wishes to visit, as the user information from the user terminal 2. The current location of the user may be a municipality selected by the user.

In Step S2, the area selector 32 determines the consultation available area based on the current location of the user. A guideline of the Ministry of Health, Labor and Welfare specifies (as of November 2021) that the user can have the online consultation in the medical facility within 30 km from the current location of the user. Thus, in the present embodiment, the area selector 32 determines an area following the guideline as the consultation available area.

In Step S3, the extraction unit 33 extracts the medical facility having the corresponding department from the medical facilities offering the online consultation in the consultation available area based on the medical facility information DB 52.

In Step S4, the extraction unit 33 displays the medical facility offering the online consultation in real time on the user terminal 2.

In Step S5, the acquisition unit 31 acquires the medical questionnaire information from the user terminal 2. If the assistance request acceptor 39 accepts the request for the assistance in preparing the medical questionnaire from the user terminal 2, the assistance request acceptor 40 transmits the assistance request to the healthcare worker terminal (not shown). The healthcare worker assists the user in preparing the medical questionnaire via chat or a web conference system, and the acquisition unit 31 acquires the prepared medical questionnaire.

In Step S6, the list generator 34 generates a list of users waiting for the online consultation in the consultation available area where the extracted medical facility is located. If the list is already prepared and notified to the medical facility terminal 3, the list generator 34 adds the user to the bottom of the list below the other users waiting for the online consultation.

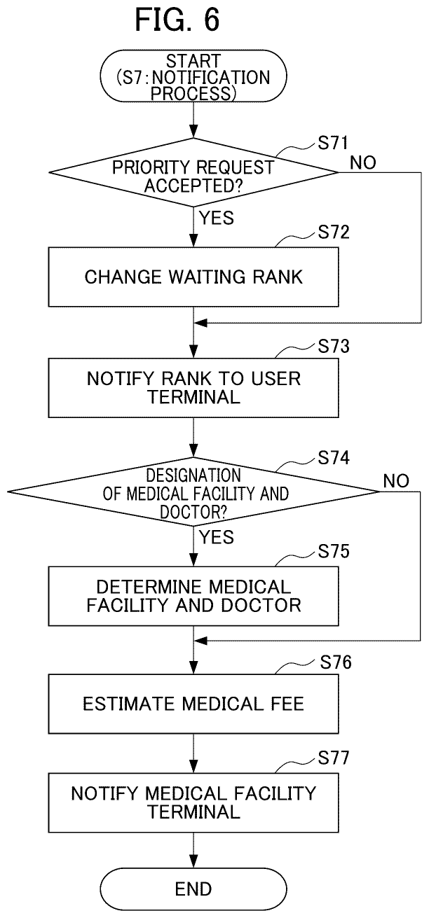

In Step S7, the notification unit 35 gives notification to the user terminal 2 and the medical facility terminal 3. The notification process in Step S7 will be described later with reference to FIG. 6.

In Step S8, the acquisition unit 31 determines whether the medical facility terminal 3 selected the patient (user). The process proceeds to Step S9 if the patient is selected, or the determination in Step S8 is repeated if not. For example, if the doctor selects the user on the top of the list (the first waiting rank), the acquisition unit 31 determines that the medical facility terminal 3 selected the patient.

In Step S9, the notification unit 35 notifies the user terminal 2 of information for the online consultation (e.g., a URL of a web conference system). The online consultation starts when the user and the doctor access the URL. The URL of the web conference system specified for each medical facility is stored in, for example, the medical facility information DB 52. However, the URL is not limited to this example, and the doctor may specify the URL when selecting the patient.

In Step S10, the acquisition unit 31 performs a process for the payment of the medical fee and the system fee with the user terminal 2.

### «Notification Process (S7)»

FIG. 6 is a flowchart of an example of the notification process in Step S7. In Step S71, the rank changer 36 determines whether the priority request is made from the user terminal 2. The process proceeds to Step S72 if the priority request is made, or to Step S73 if not.

In Step S72, in response to the priority request, the rank changer 36 raises the waiting rank of the user higher than the waiting ranks of the other users who made no priority request, if above priority request is made.

In Step S73, the notification unit 35 notifies the user terminal 2 of various pieces of information. For example, the notification unit 35 notifies the department, the registration number, and the number of waiting patients (the waiting rank). The notification unit 35 may notify a message such as “your turn is coming soon” (by displaying the message on the user terminal 2) when the number of waiting patients is reduced to a predetermined number (e.g., five). The notification unit 35 may display a screen different from the screen initially notified (displayed) when the number of waiting patients is reduced to the predetermined number. For example, a screen of the “first waiting room” may be shown first, and then a screen of the “second waiting room” may be shown when the number of waiting patients is reduced to the predetermined number.

In Step S74, the doctor selector 37 determines whether the user designated the medical facility or the doctor. The process proceeds to Step S75 if the designation is made, or to Step S76 if not.

In Step S75, the doctor selector 37 determines (selects) the medical facility or the doctor designated by the user as a notification target.

In Step S76, the fee estimator 38 estimates (calculates) the medical fee for the consultation of the user as described above based on the user information (e.g., the medical form information such as the department and the symptom).

In Step S77, the notification unit 35 notifies the medical facility terminal 3 of the departments, the number of users waiting in each department, and information on the waiting users (e.g., the name, symptom, gender, age, health insurance card information, and medical record of each user and how to receive the prescription).

### «Process of Presenting Medical Record»

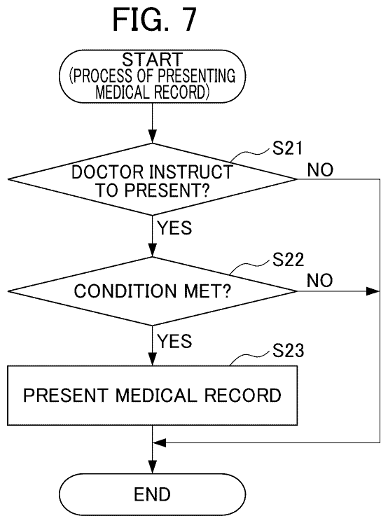

FIG. 7 is a flowchart of an example of a process of presenting the medical record. In the present embodiment, when the user (patient) shows the two-dimensional code (identification information) corresponding to the medical record to the doctor during the online consultation via the web conference system, the medical record corresponding to the identification information is shown on the medical facility terminal 3. The medical record is previously acquired from the user terminal 2 and stored in the medical record information DB 53. Examples of the information provided by the user terminal 2 in this case include the health insurance card information (images of the front and back sides of the card and the expiry date), information on an Individual Number Card (images of the front and back sides of the card and the expiry date), the results of past tests (such as images of the results of a blood test and a checkup and dates of the tests), a patient referral document (image information), how the user receives the prescription, and a password for viewing the medical record. The identification information provider 41 issues a two-dimensional code (identification information) corresponding to the medical record acquired from the user and transmits the code to the user terminal 2. The information on the expiry date may be acquired from the image information. For example, an image analyzer may further be provided to check the information on the expiry date based on the information acquired by optical character recognition (OCR) on the image using an identifier generated by a machine learning algorithm. Specifically, the image analyzer with the deep-learned identifier may further be provided to cause the identifier to recognize a text image in the image so that a text string indicating the expiry date is generated from the obtained text information. The medical record may be previously acquired from the user terminal 2 together with the medical records of family members of the user.

In Step S21, the medical record presenting unit 42 determines whether the doctor (the medical facility terminal 3) instructs to present the medical record. The process proceeds to Step S22 if the instruction is issued, or the process ends if not. The instruction to present the medical record is issued when, for example, the doctor presses a button to see the medical record of the user on a screen for referring to the medical record of the system (a patient chart storage). The instruction to present the medical record is also issued when the user shows the two-dimensional code via the web conference system during the online consultation and the doctor instructs to present the corresponding medical record based on the information on the two-dimensional code displayed on the medical facility terminal 3.

In Step S22, the medical record presenting unit 42 determines whether a condition for the presentation is met. The process proceeds to Step S23 if the condition is met, or the process ends if not. Examples of the condition include a condition in which the user whose medical record the doctor is about to view is a patient who is eligible for the online consultation by the doctor (e.g., a patient who has received the online consultation). Another condition is that the instruction is given during the online consultation. Still another condition is that a predetermined fee (a system fee) is paid by the doctor (the medical facility) when the doctor (the medical facility terminal 3) has requested the presentation of the medical record a predetermined number of times or more. The medical record presenting unit 42 determines whether any or some of the conditions are met.

In Step S23, the medical record presenting unit 42 presents the medical record to the doctor (the medical facility terminal 3). If the user permits in advance, the medical record presenting unit 42 may allow the medical facility terminal 3 to print the presented medical record (e.g., the health insurance card information).

### <Example of Screen Displayed>

### «Screen for Application of Online Consultation»

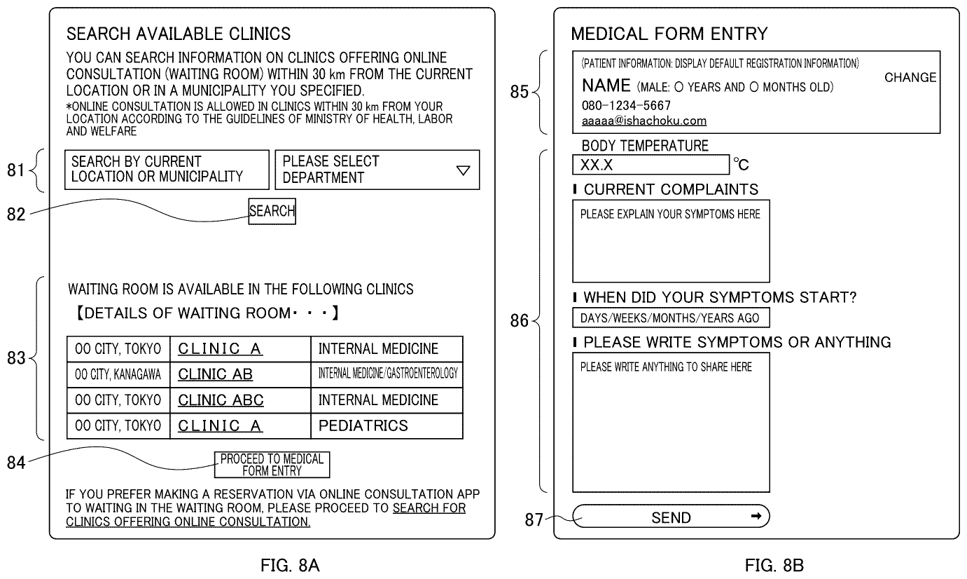

FIG. 8A and FIG. 8B are views illustrating an example of a screen for application of the online consultation shown on the user terminal 2. In the present embodiment, a transition to the screen for the application of the online consultation shown in FIG. 8A and FIG. 8B are allowed at least one of the pieces of information is stored as the medical record and the stored information has not expired.

FIG. 8A shows an example of a screen for a search of available medical facilities. Reference numeral 81 indicates a field for inputting (selecting) the location (current location or municipality) of the user and the department to visit. Reference numeral 82 indicates a search button for searching the medical facilities corresponding to the entry inputted in the reference numeral 81. Reference numeral 83 indicates a display field for displaying the result of the search based on the entry inputted in the reference numeral 81, i.e., a list of medical facilities where the user can have the online consultation. In the present embodiment, for example, the medical facilities that meet the following conditions are listed.
Within 30 km from the current location or in a selected municipality
Has a waiting room for the same department as the selected department
Currently available or active
   Reference numeral 84 indicates a button for a transition to the entry of the medical questionnaire. In the present embodiment, a screen shown in FIG. 8B is displayed when the button is pressed.

FIG. 8B shows a screen for the entry of the medical questionnaire. Reference numeral 85 indicates a field in which the user information is displayed. In the present embodiment, the name, gender, age, phone number, and e-mail address of the user are acquired from the user information and displayed. Reference numeral 86 indicates a field for the entry of the medical questionnaire information. In the present embodiment, fields for inputting the body temperature, the symptoms, time of onset of the symptoms, and any special needs are provided. Reference numeral 87 indicates a button for transmitting the inputted medical questionnaire information to the medical facility terminal 3 (e.g., via e-mail).

### «Screen of Waiting Room for Online Consultation (for Patient)»

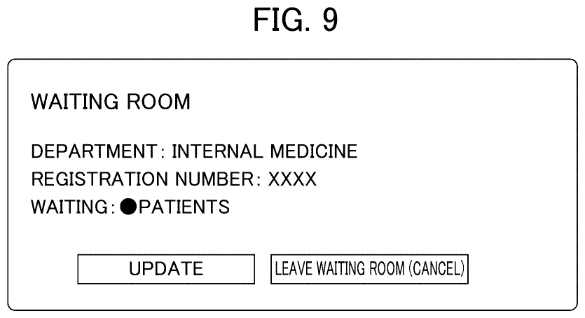

FIG. 9 is a view illustrating an example of a (patient's) screen of a waiting room (a virtual waiting room) for the online consultation displayed on the user terminal 2. In the present embodiment, the department, the registration number, and the number of waiting patients are displayed on the screen of the waiting room for the online consultation. For example, a message such as “your turn is coming soon” is shown when the number of waiting patients is reduced to a predetermined number (e.g., five) or less in the present embodiment. If the working hours of the available medical facility end while the user is waiting, the user may be informed of it. A notification setting unit may also be provided so that the setting for the notification to the user is allowed (e.g., the setting of the number of waiting patients as a trigger for the notification).

### «Screen of Waiting Room for Online Consultation (for Medical Facility)»

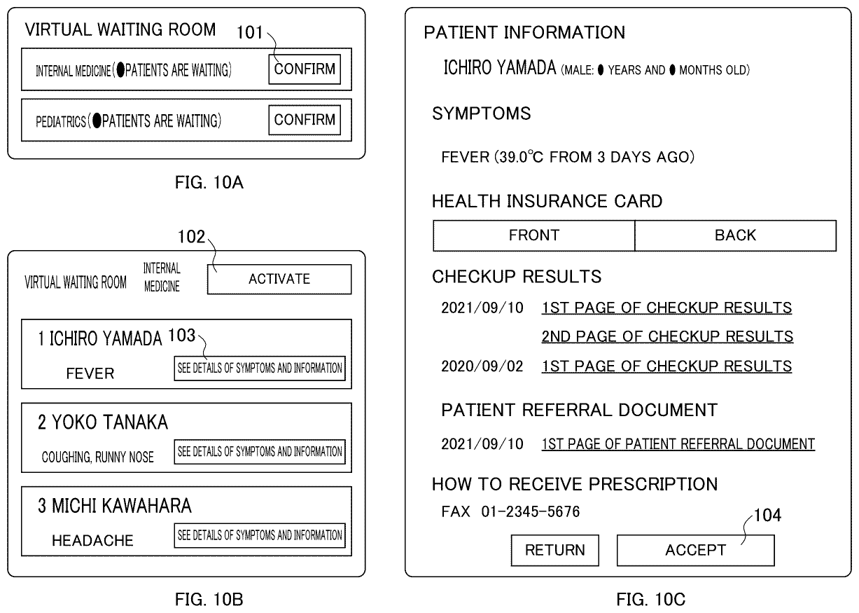

FIG. 10A to FIG. 10C are views illustrating an example of a (medical facility's) screen of a waiting room (a virtual waiting room) for the online consultation displayed on the medical facility terminal 3. FIG. 10A shows an example of the screen of the waiting room for the online consultation for the medical facility with multiple departments registered. As shown in FIG. 10A, the departments and the number of patients waiting for the consultation in each department are displayed. Reference numeral 101 indicates a button pressed to display information on the waiting patients for each department. A screen shown in FIG. 10B is displayed when the button is pressed. FIG. 10B shows a screen displaying the information on the waiting patients for each department. Reference numeral 102 indicates a button that allows the medical facility to change the online consultation in the displayed department “active (acceptable)” or “inactive (reception ended)”. Reference numeral 103 indicates a button pressed to check the details of the symptom of the user displayed and various pieces of information. A screen shown in FIG. 10C is displayed when the button is pressed. FIG. 10C shows a screen displaying the user information (patient information). In the present embodiment, the user information includes the user's personal information, medical questionnaire information, and information on the medical record (including a link to access these pieces of information). Reference numeral 104 indicates an accept button that is pressed when the doctor accepts the online consultation of the displayed user. The online consultation is accepted when the accept button is pressed. For example, when the accept button is pressed, a URL of a previously specified web conference system is transmitted from the medical facility terminal 3 to the user terminal 2. When the accept button is pressed, the URL of the previously specified web conference system may be displayed on the medical facility terminal 3 for the doctor's check. The URL may be changed to a different URL after the doctor's check. When the URL is transmitted to the user terminal, the status of the user is changed from “waiting” to “being accepted”. The user leaves the waiting room at this time. When no access to the URL is made in a predetermined period of time (e.g., 15 minutes) after the notification of the URL, the user is canceled. The doctor can cancel the user “being accepted”. If the cancellation is made a predetermined number of times, an optional penalty may be given.

### «Start Screen of Online Consultation»

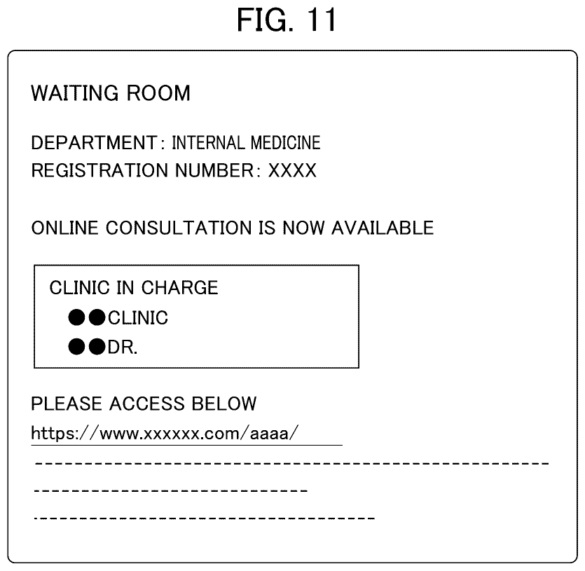

FIG. 11 is a view illustrating an example of a start screen of the online consultation displayed on the user terminal 2. In the present embodiment, the user terminal displays a message such as “online consultation is now available”, the medical facility that accepted the user, the doctor in charge, and the URL for the online consultation as shown in FIG. 19. The user accesses the URL to have the online consultation.

### Advantages of Present Embodiment

In the above-described embodiment, the online consultation is available independently of the system by using a URL of a common web conference system.

In the above-described embodiment, searching the medical facility currently available instead of making a reservation of the online consultation allows matching of the patient who seeks the online consultation and the doctor in real time.

In the above-described embodiment, the doctor is informed of the medical fee estimated from the medical form of the patient so that the doctor can select the patient taking the fee into account.

In the above-described embodiment, the priority request button (premium button) is provided so that the patient can offer incentives for the doctor to see the patient earlier.

In the above-described embodiment, the healthcare worker assists the patient in preparing the medical questionnaire, making the medical questionnaire more accurate.

In the above-described embodiment, checkup results and the information on the referral letter are inputted (registered), allowing the doctor to acquire (e.g., download) the medical record information by simply reading the two-dimensional code, for example. That is, the medical record information can be easily acquired. For example, the medical record information can be presented to the doctor when the user shows the two-dimensional code to the doctor during the online consultation or the user shows the two-dimensional code when seeing the doctor face-to-face in the medical facility. At this time, the user may set an expiry date or a user-specific password for the doctor's viewing of the medical record. The user can manage the medical record (medical information) in person and can store the medical record information independently of the doctor, allowing the user to responsibly manage his or her health conditions. In the existing regional medical cooperation system that has been introduced by various municipalities or companies, the doctor is required to input information in both of an electronic patient chart and the regional medical cooperation system with no compensation, and the patient may possibly select the other medical facility although the information is inputted. Thus, the regional medical cooperation system has not been widely accepted because it is less beneficial for the doctor to input the medical record of the patient to the system. In the above-described embodiment, the patient inputs the medical record (medical information) to the system and manages it in person. This brings no disadvantages to the doctor, contributing to the spread of the regional medical cooperation system and to the maintenance of health of many people. The doctor is obliged to provide a hardcopy of the patient chart information for free at any time at the request of the patient. Thus, the user (patient) can responsibly store (register) the medical record information for health care management.

In the above-described embodiment, the patient can timely ask doctors in all parts of the country for the online counseling. In the present situation, the online counseling is often done with an anonymous healthcare worker via chat. However, the above-described embodiment allows the patient to have the counseling with a doctor under the real name using a web conference system. This guarantees the quality of the doctor and allows a transition to the online consultation if necessary.

The embodiment of the present invention has just been described above, but the present invention is not limited to the embodiment and may be modified or improved to the extent that the purpose of the invention can be achieved.

### (Variations)

In the above-described embodiment, for example, the user at the higher waiting rank (the number of patients waiting before the user is below the predetermined number) may be informed via the user terminal 2 that the ongoing consultation by the doctor or the nurse will end soon. In this case, the type of the waiting room displayed on the user terminal 2 may be changed (e.g., from the first waiting room to the second waiting room). This allows the patient to spend the waiting time efficiently. The doctor can inform that the ongoing consultation will end soon by, for example, pressing a predetermined button.

In the above-described embodiment, an instruction may be given to inactive medical facility terminals 3 to be active. This allows requesting the inactive medical facilities (the medical facility terminals 3) to accept the online consultation when, for example, many patients are waiting for the online consultation.

The virtual waiting room of the above-described embodiment may be used as a waiting room for a nursing home (a nursing care provider). In this case, if the condition of a user in the waiting room of the nursing home is worsened, the user may be automatically connected to the virtual waiting room of the doctor.

In the above-described embodiment, a medical record during the hospital stay can be registered to the system so that the system also functions as an inpatient chart storage. The patient may ask the medical facility for a hardcopy of the patient chart every day during the hospital stay and may input the contents of the chart to the system of the present embodiment as the patient's own medical record. Thus, the medical record during the hospital stay can be used as a second opinion and can be useful for the health care after leaving the hospital. When the information on the hospital admission and discharge is acquired at the patient's will in addition to the medical information on the patient, multiple pieces of medical data dispersed and stored in multiple medical facilities only can be collected together in one place. This allows practical use of the data for the treatment in particular fields, leading to active use of research data contributing to medical development and data solving social problems.

In the above-described embodiment, it has been described that the online consultation matching system allows matching of the doctor (the first user) and the patient (the second user), but those to be matched are not limited to the doctor and the patient. For example, the matching system may be used to match any one of the first users having the following predetermined attributes and any one of the following second users.

[First users]·Those who related to any of coaching, medical representatives (MR), pharmacists, English (language) conversation lessons, fortune-telling/spiritual therapy, online dating sites, yoga/fitness, call centers (customer supports), consultation about insurance, tutors/cram school, travel agencies, counseling (including call centers for children), hostess bars, cosmetic surgery, aesthetic dentistry, companions for the elderly, trouble consultation, real estate agents, financial planners, counseling for young parents, mortgage, stock investment, asset management, lawyers, labor and social security attorneys, judicial scriveners, accountancy firms, licensed tax accountants, housing industry (construction), relationship counseling, funeral expense estimate, career change consultation, various types of estimate (check of market prices), consultation with various experts, and front end seminars

[Second users]·Users who wish to match with the first users

Specifically, the acquisition unit 31 acquires the second user information from the second user terminal operated by the second user (patient) who wishes to match with the first user (doctor) having a predetermined attribute. The second user information includes background information such as the second user's medical record, background, and usage history of a predetermined service in addition to the user information described above. For the online consultation, for example, the background information includes a personal health record (PHR), a nursing care record, and online eligibility check information (drug information). The extraction unit 33 extracts the first user who is matchable with the second user based on the second user information. As a possible example, the notification unit 35 may output a list of the second users matched and the second user information to the terminal of the first user.

In the above-described embodiment, the area corresponding to the first user is limited based on the current location of the user (the second user), but the present invention is not limited to this example. For example, the user can receive (match with) the service without selecting a service provider (with no limitation of job categories), i.e., the first user can be selected by the extraction unit, anywhere in the world. Specifically, the designation of the area is not required for the online consultation, for example. Thus, the doctor can specify the area or select the area from all parts of the country, and the patient can also specify the area or select the area from all parts of the country.

In the above-described embodiment, for the other online services than the online consultation, the first user may be an online service provider, a facility that offers a service, and a service recipient (e.g., service industries, qualified professionals, insurance companies, consulting services, educational services, language schools, fortune-telling services, and call centers).

In the above-described embodiment, the matching system can be applied not only to the matching between the service provider and the service recipient, but to the matching between individuals. For example, the system can be applied to the matching between a man who meets predetermined conditions and a woman looking for a man who meets the predetermined conditions. In this case, the area is limited to raise the possibility of the matching with the desired counterpart.

In the above-described embodiment, the online consultation is done between two persons, namely, the doctor and the patient, but the present invention is not limited to this example. For example, three or more persons can speak (talk) online. In this case, examples of a combination of the first user, the second user, and the third person (the third user) include those described below.

[First user, second user, third user]·[a doctor, rural parents (care recipients), a son in an urban area], [a primary care doctor (e.g., a family doctor), a patient, an attending doctor (e.g., a specialist)], [a doctor, a patient (a foreigner), an interpreter]

The third user can participate in the online talk between the matched first and second users when one or both of the first and second users share the link (the link for the online talk). The third user is not limited to a single person and may be two or more persons.

In the above-described embodiment, time for the online talk (e.g., the online consultation) is not limited, but a timer may be set to limit the time. For example, the user may be charged by time.

In the above-described embodiment, the first user (e.g., a doctor) may be able to change to another doctor in the middle of the online talk.

In the above-described embodiment, the patient (the second user) is not allowed to designate the first user (the doctor) for the first time of use, but the designation may be allowed after the second time of use. Specifically, the acquisition unit 31 does not accept a designation instruction from the second user terminal to designate a predetermined user among the first users when the second user wishes to match with the first user for the first time. The acquisition unit 31 is allowed to accept the designation instruction from the second user terminal to designate a predetermined user among the first users when the second user wishes to match with the first user for the second time or more. The extraction unit 33 extracts the predetermined user as the first user who is matchable when the designation instruction is accepted. This allows the second user (e.g., the patient) to match again with the first user (e.g., the doctor) who matched before, improving convenience for the second user.

In the above-described embodiment, a fee for the designation of the first user may be charged to the second user who uses the system for the second time or more to match with the first user.

In the above-described embodiment, the first user (a service provider) may block (decline the designation by) the second user (a service recipient) and remove the first user from the designation list (the list indicating that the first user is designated). Specifically, the acquisition unit 31 is allowed to accept an instruction to decline the designation instruction from the first user terminal of the designated predetermined user when the designation instruction is accepted. When the instruction to decline the designation is issued, the extraction unit 33 does not extract the predetermined user as the first user who is matchable.

In the above-described embodiment, the second user may previously enter what the second user wishes to discuss (the second user information) like a medical questionnaire, and the first user may select the second user based on the entry.

For the online consultation in the above-described embodiment, AI may automatically determine and display information relevant to the name of a predetermined disease selected by the first user (the doctor) on the first user terminal. Examples of the relevant information include the effect of drugs, for example. In the above-described embodiment, AI may output information relevant to the predetermined results of diagnosis during the online consultation. For example, an alert may be outputted when abnormality is found from the noises from the heart (heart sounds).

In the above-described embodiment, the priority (the rank displayed) of the patient in the virtual waiting room may be changed based on various factors. For example, the rank may be raised based on a predetermined formula of relevancy (e.g., a side effect rate x relevancy). When the patient is having asthma as a side effect of something, the patient can be treated with priority. When the priority is changed automatically, even an inexperienced doctor can diagnose the patients in the suitable order.

In the above-described embodiment, the notification unit 35 may output information on a predetermined social networking service to the second user terminal. This allows, for example, an MR to function as a call center or a predetermined subject to function as an SOS call center.

The medical record (the patient chart storage) of the above-described embodiment may further include a nursing care record of the patient. For example, the nursing care record is created by a visiting nurse or a care manager.

The medical record (the patient chart storage) of the above-described embodiment may allow, not only the doctors, but caregivers, to access and see the medical record (medical information) and may allow, not only the caregivers, but doctors, to access and see the medical record (nursing care information).

The medical record (the patient chart storage) of the above-described embodiment may acquire check information on the patient (information for checking the eligibility for the online consultation). This information can be used as health information related to the first online consultation.

In the medical record (the patient chart storage) of the above-described embodiment, how the original data is displayed may be changed, for example, the data may be color-coded by department, or the histories of the prescription for high blood pressure are extracted.

In the medical record (the patient chart storage) of the above-described embodiment, the trade name of a particular drug may be replaced with the name of a product corresponding to the drug (e.g., Olopatadine) for promoting the use of generic drugs. This hides the name of the originator drug, promoting the use of the generic drugs.

In the medical record (the patient chart storage) of the above-described embodiment, drugs contraindicated for combined use may be shown.

In the medical record (the patient chart storage) of the above-described embodiment, the names of pharmaceutical companies may be stored together with the information on the drugs. Thus, an inquiry to the pharmaceutical company about usage of the drug can be done without reference to a drug dictionary.

The medical record (the patient chart storage) of the above-described embodiment may provide various functions in cooperation with a predetermined dictionary database. For example, the medical record may follow and store drug information (DI) updated every week. The number of side effects increases with the increase of side effect reports, and keeping up with the latest information is required.

### (Others)

For example, the processes described above may be performed by hardware or software. That is, the above-described functional configuration is merely an example and does not limit the present invention. Specifically, it is sufficient for the information processing device or system to have the function that can achieve the series of processes described above, and the functional blocks used to achieve the function are not particularly limited to the examples described above. The locations of the functional blocks are not particularly limited to the examples shown in FIG. 4 and may be determined as appropriate. For example, the functional blocks in the server may be transferred to a different device. Conversely, the functional blocks in a different device may be transferred to the server. A single functional block may be constituted of hardware alone, software alone, or a combination of the hardware and the software.

When the software is used to perform the series of processes, programs constituting the software are installed in a computer via a network or a storage medium. The computer may be installed in exclusive hardware. Alternatively, the computer may be a computer with various programs installed to achieve various functions, for example, a server, a general-purpose smartphone, or personal computer.

A storage medium including such programs is constituted of not only a removable medium which is not shown and is distributed to a user separately from the main device to provide the program, but also a storage medium installed in advance in the main device and provided to the user. The programs can be distributed via a network, and the storage medium may be mounted on a computer that is connected or connectable to the network or accessible to the network.

The step of writing the program in the storage medium includes processes performed chronologically in order, and also include processes that are not necessarily performed chronologically in order but performed in parallel or individually. In this specification, the term “system” designates an entire apparatus including multiple devices and multiple instruments.

In other words, a parts supply system to which the present invention is applied can take the form of various embodiments described below. Specifically, the present invention is applicable to (1) an information processing device including: an acquisition unit (e.g., the acquisition unit 31) that acquires second user information on at least one second user who wishes to match with at least one first user having a predetermined attribute from a second user terminal operated by the second user;
an extraction unit (e.g., the extraction unit 33) that extracts the at least one first user who is matchable based on the second user information; and
an output unit (e.g., the notification unit 35) that outputs a list of the at least one second user matched and the second user information to a terminal of the first user. This allows a person to match with a person having a predetermined attribute in real time.

(2) The information processing device may further include: an area selector (e.g., the area selector 32) that determines a consultation available area where an online consultation with the first user is available for the second user based on the second user information:
a storage (e.g., the medical facility information DB 52) that stores medical facility information on medical facilities offering the online consultation; and
a list generator (e.g., the list generator 34) that generates a predetermined list. The extraction unit may extract at least one medical facility offering the online consultation in the consultation available area in real time based on the medical facility information. The list generator may generate a list of the second users waiting for the online consultation in the consultation available area where the extracted medical facility is located. The output unit may output the list and the second user information to a medical facility terminal used by the extracted medical facility. This allows matching between the user who seeks the online consultation and the medical facility in real time.

(3) The second user information may include location information on the second user, and the area selector may determine an area within a predetermined distance from a location indicated by the location information on the second user as the consultation available area. This allows an easy search of the medical facilities within 30 km, which is currently specified for the online consultation by the Ministry of Health, Labor and Welfare.

(4) The output unit may output a waiting rank of the second user for the online consultation in the medical facility in the consultation available area to the second user terminal.

(5) The output unit may output a notification indicating that the consultation for the second user will occur soon when the waiting rank reaches a predetermined rank. This allows the user to spend the waiting time efficiently.

(6) The acquisition unit may accept a priority request for a priority consultation from the second user terminal. The information processing device may further include a rank changer (e.g., the rank changer 36) that raises, in response to the priority request, the waiting rank of the second user who made the priority request that is higher than a waiting rank of another second user who has not made a priority request. This allows the patient to offer incentives to the doctor for the priority online consultation.

(7) The acquisition unit may accept designation of a doctor in charge of the online consultation from the second user terminal. The information processing device may further include a doctor selector (e.g., the doctor selector 37) that determines a doctor who will be in charge of the online consultation with the second user who made the designation of the doctor in response to the designation of the doctor. This allows the patient to request the desired doctor for the online consultation.

(8) The information processing device may further include a medical fee estimator (e.g., the fee estimator 38) that estimates a medical fee for the consultation of the second user based on the second user information. The output unit may output the estimated medical fee to the medical facility terminal. This can present information that the doctor refers to when selecting the patient.

(9) The second user information may include medical form information on a symptom of the second user, and the information processing device may further include: an assistance request acceptor (e.g., the assistance request acceptor 39) that accepts a request to assist the second user in preparing the medical form information; and an assistance requestor (e.g., the assistance requestor 40) that requests a predetermined healthcare worker to assist the second user in preparing the medical form information via a healthcare worker terminal operated by the predetermined healthcare worker when the assistance request from the second user is accepted. This allows the preparation of an accurate medical questionnaire.

(10) The acquisition unit may acquire a medical record transmitted by the second user. The information processing device may further include: a medical record storage (e.g., the medical record information DB 53) that stores the medical record:
an identification information provider (e.g., the identification information provider 41) that gives identification information to the medical record; and
a presenting unit (e.g., the medical record presenting unit 42) that presents the medical record corresponding to the identification information to the medical facility terminal based on the identification information. Thus, the user can acquire and store the medical record in place of the doctor, saving the doctor time and effort.

(11) The presenting unit may present the medical record to the medical facility terminal on a condition that a predetermined fee is paid by the medical facility when the medical facility terminal has requested the presentation of the medical record a predetermined number of times or more. Thus, the presentation of the medical record to the user, which is a fee-charging option, can be free up to the predetermined number of times, allowing the function of presenting the medical record to spread widely.

(12) The extraction unit may extract a medical facility that offers online counseling in real time based on the medical facility information, the list generator generates a list of second users waiting for the online counseling, and the output unit may output the list of the second users waiting for the online counseling and the second user information to a medical facility terminal used by the extracted medical facility offering the online counseling. This allows the doctor to provide the counseling to the patients from all parts of the country in the doctor's spare time. When the patient having the online counseling with the doctor is eligible for the online consultation (e.g., the patient lives in an area within 30 km from the medical facility), the doctor can switch the online counseling to the online consultation.

(13) The acquisition unit may not accept a designation instruction from the second user terminal to designate a predetermined user among the first users when the second user wishes to match with the first user for the first time, and the acquisition unit may be allowed to accept the designation instruction from the second user to designate the predetermined user among the first users when the second user wishes to match with the first user for a second time or more. The extraction unit may extract the predetermined user as the first user who is matchable when the designation instruction is accepted. This allows the second user (e.g., the patient) to be rematched with the first user (e.g., the doctor) with whom he or she was previously matched, thereby improving convenience for the second user.

(14) The acquisition unit may be allowed to accept an instruction to decline the designation instruction from the first user terminal of the designated predetermined user when the designation instruction is accepted, and the extraction unit may not extract the predetermined user as the first user who is matchable when the instruction to decline the designation instruction is accepted. This allows the first user to decline the designation due to the circumstances of the first user, improving convenience for the first user.

(15) The output unit may output information for having conversation via a predetermined network to a first user terminal operated by the first user, the second user terminal operated by the second user who matched with the first user, and a third user terminal operated by a third user designated by the first or second user. This allows not only the first and second users but a third person designated by the first or second user to talk together.

(16) The second user information may include background information on the second user. This allows the first user to know the background information on the second user before talking with the second user.

(17) The output unit may output information on a predetermined social networking service to the second user terminal. This can improve convenience for the second user.

(18) The present invention can be implemented as a method for controlling an information processing device including a computer that performs the steps of: acquiring second user information on at least one second user who wishes to match with at least one first user having a predetermined attribute from a second user terminal operated by the second user; extracting the at least one first user who is matchable based on the second user information; and outputting a list of the at least one second user matched and the second user information to a terminal of the first user.

(19) The present invention can also be implemented as a computer program that causes a computer to perform the steps of: acquiring second user information on at least one second user who wishes to match with at least one first user having a predetermined attribute from a second user terminal operated by the second user; extracting the at least one first user who is matchable based on the second user information; and outputting a list of the at least one second user matched and the second user information to a terminal of the first user.

EXPLANATION OF REFERENCE NUMERALS

| | | |
|---|---|---|
| 1: Server | 2: User terminal | 3: Medical facility terminal |
| 11: CPU | 18: Storage | 19: Communication unit |
| 31: Acquisition unit | 32: Area selector | 33: Extraction unit |
| 34: List generator | 35: Notification unit | 36: Rank changer |
| 37: Doctor selector | 38: Fee estimator | 39: Assistance request |
| | | acceptor |
| 40: Assistance | 41: Identification | 42: Medical record |
| requestor | information provider | presenting unit |
| 51: User | 52: Medical facility | 53: Medical record |
| information DB | information DB | information DB |
| 54: Company | | |
| information DB | | |

1. An information processing device, comprising:
an acquisition unit that acquires second user information on at least one second user who wishes to match with at least one first user having a predetermined attribute from a second user terminal operated by the second user;
an extraction unit that extracts the at least one first user who is matchable based on the second user information; and
an output unit that outputs a list of the at least one second user matched and the second user information to a terminal of the first user.

2. The information processing device according to claim 1, further comprising: an area selector that determines a consultation available area where an online consultation with the first user is available for the second user based on the second user information;
a storage that stores medical facility information on medical facilities offering the online consultation; and
a list generator that generates a predetermined list, wherein
the extraction unit extracts at least one medical facility offering the online consultation in the consultation available area in real time based on the medical facility information,
the list generator generates a list of the second users waiting for the online consultation in the consultation available area where the extracted medical facility is located; and
the output unit outputs the list and the second user information to a medical facility terminal used by the extracted medical facility.

3. The information processing device according to claim 1, wherein the second user information includes location information on the second user, and
an area selector determines an area within a predetermined distance from a location indicated by location information on the second user as a consultation available area.

4. The information processing device according to claim 1, wherein the output unit outputs a waiting rank of the second user for the online consultation in the medical facility in the consultation available area to the second user terminal.

5. The information processing device according to claim 4, wherein the output unit outputs a notification indicating that the consultation for the second user will occur soon when the waiting rank reaches a predetermined rank.

6. The information processing device according to claim 4, wherein the acquisition unit accepts a priority request for a priority consultation from the second user terminal, and
the information processing device further includes a rank changer that raises, in response to the priority request, the waiting rank of the second user who made the priority request that is higher than a waiting rank of another second user who has not made a priority request.

7. The information processing device according to claim 1, wherein the acquisition unit accepts designation of a doctor in charge of the online consultation from the second user terminal, and
the information processing device further includes a doctor selector that determines a doctor who will be in charge of the online consultation with the second user who made the designation of the doctor in response to the designation of the doctor.

8. The information processing device according to claim 1, further comprising a medical fee estimator that estimates a medical fee for the consultation of the second user based on the second user information, wherein
the output unit outputs the estimated medical fee to a medical facility terminal.

9. The information processing device according to claim 1, wherein the second user information includes medical form information on a symptom of the second user, and
the information processing device further includes: an assistance request acceptor that accepts a request to assist the second user in preparing the medical form information; and
an assistance requestor that requests a predetermined healthcare worker to assist the second user in preparing the medical form information via a healthcare worker terminal operated by the predetermined healthcare worker when the assistance request from the second user is accepted.

10. The information processing device according to claim 1, wherein the acquisition unit acquires a medical record transmitted by the second user, and
the information processing device further includes: a medical record storage that stores the medical record;
an identification information provider that gives identification information to the medical record; and
a presenting unit that presents the medical record corresponding to the identification information to the medical facility terminal based on the identification information.

11. The information processing device according to claim 10, wherein the presenting unit presents the medical record to the medical facility terminal on a condition that a predetermined fee is paid by the medical facility when the medical facility terminal has requested the presentation of the medical record a predetermined number of times or more.

12. The information processing device according to claim 1, wherein the extraction unit extracts a medical facility that offers online counseling in real time based on the medical facility information,
a list generator generates a list of second users waiting for the online counseling, and
the output unit outputs the list of the second users waiting for the online counseling and the second user information to a medical facility terminal used by the extracted medical facility offering the online counseling.

13. The information processing device according to claim 1, wherein
the acquisition unit is not allowed to accept a designation instruction from the second user terminal to designate a predetermined user among the first users when the second user wishes to match with the first user for a first time,
the acquisition unit is allowed to accept the designation instruction from the second user to designate the predetermined user among the first users when the second user wishes to match with the first user for a second time or more, and
the extraction unit extracts the predetermined user as the first user who is matchable when the designation instruction is accepted.

14. The information processing device according to claim 13, wherein the acquisition unit is allowed to accept an instruction to decline the designation instruction from the first user terminal of the designated predetermined user when the designation instruction is accepted, and
the extraction unit is not allowed to extract the predetermined user as the first user who is matchable when the instruction to decline the designation instruction is accepted.

15. The information processing device according to claim 1, wherein the output unit outputs information for having conversation via a predetermined network to a first user terminal operated by the first user, the second user terminal operated by the second user who matched with the first user, and a third user terminal operated by a third user designated by the first or second user.

16. The information processing device according to claim 1, wherein the second user information includes background information on the second user.

17. The information processing device according to claim 1, wherein the output unit outputs information on a predetermined social networking service to the second user terminal.

18. A method for controlling an information processing device including a computer that performs the steps of: acquiring second user information on at least one second user who wishes to match with at least one first user having a predetermined attribute from a second user terminal operated by the second user;
extracting the at least one first user who is matchable based on the second user information; and
outputting a list of the at least one second user matched and the second user information to a terminal of the first user.

19. A non-transitory computer readable medium storing a computer program that causes a computer to perform the steps of: acquiring second user information on at least one second user who wishes to match with at least one first user having a predetermined attribute from a second user terminal operated by the second user;
extracting the at least one first user who is matchable based on the second user information; and
outputting a list of the at least one second user matched and the second user information to a terminal of the first user.
